(19) United States
(12) Patent Application Publication
(10) Pub. No.: US 2010/0222753 A1
(43) Pub. Date: Sep. 2, 2010

(54) Patch With Hydrophilic Adhesive

(76) Inventors: Bertrand Dupont, Aix En Provence (FR); Nathalie Donne, Allauch (FR)

Correspondence Address: JONES DAY, 222 EAST 41ST ST, NEW YORK, NY, 10017, US

(21) Appl. No.: 12/680,893

(22) PCT Filed: Sep. 30, 2008
(86) PCT No.: PCT/FR08/51749

§ 371 Date: Mar. 30, 2010

(30) Foreign Application Priority Data
Oct. 1, 2007 (FR) 0757970

(51) Int. Cl. A61F 13/02; B05D 7/00

(52) U.S. Cl. 604/307; 427/2.31

(57) ABSTRACT

The invention concerns a patch (1) for application to the skin, comprising:
a hydrophilic powder (2) acquiring an adhesive property in the presence of water;
a backing (4) having an application side on which the hydrophilic powder (2) is deposited.

The present invention generally concerns devices for application to the skin. The invention more particularly concerns products and methods allowing better skin attachment of devices such as skin patches or transdermal devices intended to interact with the skin for its hydration or to facilitate the skin absorption of biologically active substances. It also concerns a method for manufacture of such devices, and their uses in therapy, diagnosis and/or cosmetics for example.

Cutaneous or percutaneous absorption corresponds to the transfer of a substance through the skin, typically from the external medium to the blood. This absorption is generally defined as being the sum of two phenomena: penetration of molecules into the layers of the skin, followed by reabsorption by circulating blood or lymph from the papillary dermis and then the deep dermis. The penetration step is physically a passive diffusion through each or some of the structures of the tegument: stratum corneum, Malpighi layer, dermis and skin appendages. Penetration, in its broadest meaning, does not necessarily involve diffusion through the whole of the structures of the tegument, notably the deepest ones, but should preferably allow exposure of the substance to the immune or circulation system. Once absorbed, the substance is generally distributed in the organism, then after being metabolized, or not, it is eliminated. The steps following percutaneous absorption are normally similar to those typical for any other route of administration.

The stratum corneum forms the most efficient barrier against penetration of a substance: anatomically, the substance may penetrate via two routes: one via the intercellular spaces of the stratum corneum and through the horny cells themselves, the other via the skin appendages. It has been mentioned that the passage of hydrophilic active ingredients into the stratum corneum is facilitated when these compounds are contained in a moist atmosphere. In the case of a vaccine, once a substance (antigen, immunogen, adjuvant, etc.) has passed through the stratum corneum, it is capable of meeting immunologically competent cells and, in particular, the antigen-presenting cells such as Langerhans cells which play an essential role in the immunological reaction of the body. Facilitating the initial passage of the antigen through the stratum corneum imparts greater efficacy to epicutaneous immunization. With regard to a patch intended to administer a molecule for systemic treatment, if the barrier efficacy of the stratum corneum is temporarily reduced, this will open the way to the transcutaneous administering of active ingredients having high molecular weights, and generally will improve the rate of administration of active ingredients (proteins, peptides, chemical molecules, nucleic acid, etc.).

Different types of patch have been proposed in the literature for the cutaneous transfer of active substances. Generally, these patches are “adhesive”, i.e., they are provided with a glue or adhesive, typically of acrylic type, to hold the patch close to the skin.

For example, document US2004/137004 concerns a transdermal patch possibly comprising different formulations and an acrylic adhesive in the form of a hydrogel. Document US2006/002949 concerns a transcutaneous immunization method without any adjuvant in which an antigen in powder form can be used. Document US2006/147509 concerns a device for the transdermal delivery of an immunogen, comprising a delivery system containing saponin and sterol and an occlusive vehicle. Document WO00/43058 concerns a transcutaneous method to administer an active ingredient in the form of accelerated particles. The method uses either an injection by means of a PowderJect™ device, or a patch held in position by an athlete's band. Document WO02/07325 concerns a patch for transcutaneous immunization and document WO2006/007366 concerns fusion proteins for transcutaneous immunization. In these documents, the antigen can be used in powder or liquid form, either alone or mixed with an adjuvant. If an adhesive is used to hold the patch in place, this may be either a resin or an aqueous adhesive.

Patches are also known to be used to hydrate the skin. In this case, the patch does not necessarily contain an active substance, but its application, notably in occlusive format, creates a hydrating region in contact with the skin. Said patches are mainly intended for the cosmetic sector.

The prior art patches have disadvantages, in particular if the desired effect requires strong patch adhesion obtained with a conventional adhesive (e.g. acrylic). When the patch is removed, the separation between the adhesive surface and the skin may indeed lead to irritation and cause pain. Also, the use of aqueous adhesives may lead to deterioration of the active ingredient on the patch, and hence to a short shelf life. A compromise must therefore be found regarding the adhering property of the adhesive surface, in order to limit pain during patch removal, whilst guaranteeing holding the patch on the skin. This adhesion is relatively difficult to determine precisely, since it may vary with the storage time of the patch before its application.

The invention sets out to solve one or more of these disadvantages. The invention provides a patch with hydrophilic adhesive for application to the skin, characterized in that the adhesion of the patch to the skin is ensured by a hydrophilic powder which acquires an adhesive property in the presence of water. This characteristic of the invention ensures improved adhesion of the device when it is applied, easy storage, no loss of adhesiveness in relation to storage time, stability of the active ingredient (maintained in dry form) and better penetration into the skin of any active substance due to the prior hydration (natural or artificial) of the skin necessary for transforming the hydrophilic powder into an adhesive gel.

The advantages of this powder adhesive are the following:
Compared with use of a hydrogel: patches using a hydrogel require a substantial quantity of hydrogel to be deposited on the patch, generally over the entire surface of the patch, which means that it is difficult to handle; said patch cannot be worn on the skin like a conventional transdermal patch, under clothing for example; additionally, storage of the hydrogel, which is moist by definition, in its pack before application, is limited (limited storage time); finally the use of a hydrogel prevents any active ingredient from being presented in dry form which may be required for storage in some cases because the water contained in the hydrogel will moisten the active ingredient.
Compared with the use of acrylic or silicon adhesives: Some devices for skin application, in order to obtain deep, local hyperhydration of the skin underneath the device (for example to facilitate passage of the active ingredient) or to obtain better dissolution of the dry active ingredient deposited on the inner surface (facing the skin) of the device, necessitate prior wetting of the skin; in this case, the use of a hydrophilic powder which acquires adhesive properties in the presence of water, allows immediate adhesion of the device onto wet skin, which is not possible with an adhesive of acrylic or silicon type.

A particular object of the invention therefore concerns a patch for application to the skin, comprising:
a hydrophilic powder acquiring an adhesive property in the presence of water; and
a backing having an application side on which the hydrophilic powder is deposited.

In the patch of the invention, the hydrophilic powder, in dry form, therefore acquires an adhering property in the presence of water, thereby permitting the patch to be held firm when applied to skin which has advantageously been previously moistened.

The hydrophilic powder can be deposited on all or part of the application side of the patch, over a sufficient area to permit adhesion. According to one variant, the hydrophilic powder is arranged on the periphery of the application side of the backing.

According to another variant, the hydrophilic powder forms spots distributed over the application side of the backing.

According to a further variant, the hydrophilic powder is deposited on the entire surface of the application side of the backing.

According to a further variant, the patch also comprises a biologically active substance.

According to one variant, the substance is a powder mixed with the hydrophilic powder.

According to another variant, the substance is deposited on area(s) of the application side of the backing that is/are distinct from the hydrophilic powder.

According to another variant, the hydrophilic powder contains water-soluble polymer chains.

According to one variant, the hydrophilic powder contains cellulose gum, ethylcellulose, hydroethylcellulose, a copolymer of N-vinyl-2-Pyrrolidone and vinyl acetate, or a polymer of 1-vinyl-2-pyrrolidone.

According to another variant, the patch further comprises a reservoir containing an aqueous solution positioned above the hydrophilic powder.

According to another variant, the patch comprises a sealed packaging impervious to water vapour in which the backing and the hydrophilic powder are included.

According to a further variant, the backing is in the shape of a comma.

The invention also relates to a method of manufacture of a patch which comprises applying a hydrophilic powder, acquiring an adhesive property in the presence of water, onto the application side of a backing. The backing may be of varied shape and type and notably may be flat, or not, of polymer, plastic, metal, textile and/or biological material, solid or semi-solid, etc.

According to one variant, the hydrophilic powder is applied over all or part of the surface of the application side.

According to another variant, the hydrophilic powder is applied to the periphery of the application side or in the form of spots distributed over the surface of the application side of the backing.

According to another variant, the method further comprises a step of applying a biologically active substance onto the surface of the backing.

According to another variant, the substance is applied in the form of a powder in a mixture with the hydrophilic powder.

According to another variant, the substance and the hydrophilic powder are successively applied.

According to one variant, the powder and/or the substance are held on the backing by forces of electrostatic type.

A further object of the invention concerns a method for cutanous application of a substance to the skin, comprising application of a patch such as that defined above to the skin of a subject, the application step being preceded, or not, by a pre-hydration step of the skin.

A further object of the invention concerns a method to hydrate the skin of a subject comprising application of a patch such as that defined above to an area of skin to be hydrated.

A further object of the invention concerns the use of a patch such as that defined above for the application of a substance to the skin and/or to deliver a substance via the epi- or transcutaneous route to a subject, notably a mammal, in particular a human being (for example a child or adult). The patch may notably be used for the vaccination of subjects, for desensitization of subjects, or for the delivery of any active substance such as biologically active and/or antigenic (poly)peptides, in particular. The invention also concerns corresponding methods comprising the application of a patch of the invention to the skin of a subject, this application step being preceded, or not, by a pre-hydration step of the skin.

The patch can be used to deliver any active substance. Therefore, one particular object of the invention resides in a method to deliver an active substance to a subject, comprising (i) applying a patch according to the invention to the skin of a subject, the patch containing said active substance and (ii) holding the patch firm for a period of time to allow transfer of the substance into the skin. The substance is typically of polypeptide type such as a hormone, cytokine, growth factor, trophic factor, etc.

The substance contained in the patch can be formulated in any adapted vehicle or excipient and can be in solid form (powder), liquid form, etc.

For example, the patch can be used to vaccinate subjects against any pathogen. Therefore one particular object of the invention resides in a method to vaccinate a subject against a pathogen, comprising (i) applying a patch according to the invention to the skin of a subject, the patch containing an antigen specific to said pathogen, and (ii) holding the patch firm for a period of time permitting transfer of the antigen into the skin. The pathogen may be of varied type (virus, bacterium, parasite etc.) and the antigen is typically of polypeptide or lipid type.

The patch may also be used to desensitize subjects against allergens. Therefore, one particular object of the invention resides in a method of desensitization of a subject against an allergen, comprising (i) applying a patch according to the invention onto the skin of a subject, the patch containing an antigen specific to said allergen, and (ii) holding the patch firm for a period of time permitting transfer of the antigen into the skin.

In the above methods, the application of the patch is preferably preceded by hydration (or moistening) of the skin.

Other characteristics and advantages of the invention will become clearly apparent from the following description given by way of indication and not limitation, with reference to the appended drawings in which:

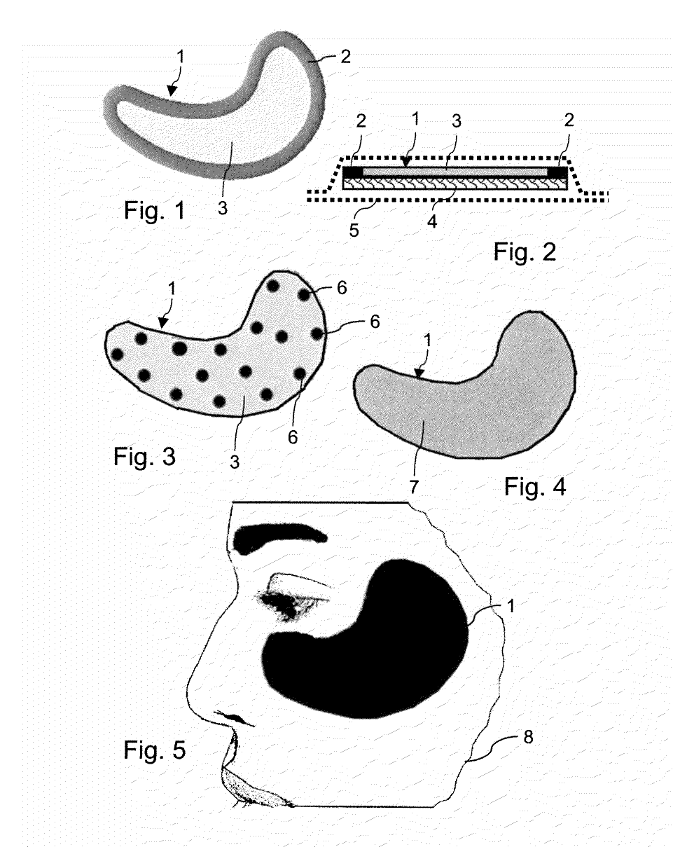

FIG. 1 is a top view of the adhesive side of a patch according to the invention;

FIG. 2 is a side, cross-sectional view of the patch of FIG. 1 placed inside a pack;

FIG. 3 is a top view of the adhesive side of one variant of patch;

FIG. 4 is a top view of the adhesive side of another variant of patch;

FIG. 5 is a side view of a user's face on which the patch is applied.

The invention proposes forming the fixing adhesive of a patch by means of a hydrophilic powder which acquires an adhesive property in the presence of water. The invention is adapted to any type of patch, i.e., any device which can be applied to an area of a subject's skin for placing it in contact with a substance or to create a hydrating region. The device typically comprises a backing which may be of varied shape or type, notably flat, or not, of polymer, plastic, metal, textile and/or biological material, solid or semi-solid, etc.

Different types of devices can be used to carry out the present invention. In particular, the patches described in documents EP 1 367 944 or FR2,866,553 can be cited. Such patches typically comprise a polymer material (e.g. a film of plastic material) on the surface of which a powder can be fixed via electrostatic forces.

Another type of patch which can be used to carry out the invention is for example a patch such as described in application EP1356821, which mentions the successive use of an adhesive film intended to remove a part of the stratum corneum, and a patch to make a vaccine penetrate via the epicutaneous route.

Other patch systems such as cups etc. can be used according to the present invention.

FIGS. 1 and 2 illustrate a first embodiment of a patch 1 according to the invention, intended in this case for the diffusion of an active substance into the body of the user. The patch 1 comprises a backing 4. The backing 4 has an application side on which a hydrophilic powder 2 is deposited. A substance 3 is also advantageously deposited on the application side. The substance 3 is intended to enter into the epidermis in a manner known per se. The hydrophilic powder 2 is present in dry form and acquires its adhering property in the presence of water. The hydrophilic powder 2 is then converted to a gel which adheres to the skin, thus enabling adhesion of the patch 1.

Therefore, the adhesive property of the patch 1 at the time of application is not decreased by any length of storage time. Additionally, the adhesive property in the presence of water is relatively reduced, facilitating pain-free removal of the patch 1.

Any hydrophilic powder 2 can be used which transforms into an adhesive when in contact with a moist medium and enabling the adhesion and holding of the patch 1 firm on the skin. The hydrophilic powder 2 is preferably a biocompatible and inert powder, it therefore does not interact with the active ingredients of the substance 3 and also does not give rise to any adverse reactions.

The hydrophilic powder 2 is advantageously a hydrocolloid or hydrogel, i.e., it contains water-soluble polymer chains. The hydrophilic powder may in particular contain the following compounds: cellulose gum (notably the gum distributed under the trade name Blanose), ethylcellulose (notably distributed under the trade name Ethocel), hydroethylcellulose (notably distributed under the trade name Natrosol), a copolymer of N-vinyl-2-Pyrrolidone and vinyl acetate (notably distributed under the trade name Plasdone S-630), and/or a polymer of 1-vinyl-2-pyrrolidone (notably distributed under the trade name Plasdone K-29/32).

The addition of water can be achieved by various means, whether natural (sweating, perspiration of the user at the time of application) or artificial (water provided externally, e.g., by means of a spray, wash flannel, cotton, hydrating gel or cream advantageously containing more than 80% water). Artificial addition of water may also be obtained by providing the patch 1 with a reservoir (not illustrated) and positioned above the hydrophilic powder 2. The reservoir then includes an aqueous solution. The reservoir may be in the form of a tear-off envelope to impregnate the hydrophilic powder 2.

In the example shown in FIG. 1, the hydrophilic powder 2 is joined to the periphery of the backing 4. The hydrophilic powder 2 is arranged on the periphery of the application side of the backing 4 and surrounds a surface coated with the substance 3. The periphery of the application side of the backing 4 is therefore used for fixing it to the skin, whilst the central portion of the application side is used to place the substance 3 close against the user's skin.

As illustrated in FIG. 2, the patch 1 advantageously comprises a sealed packaging 5 in which the backing 4 is arranged. This packaging 5 is used to keep the hydrophilic powder 2 dry before use of the patch 1. The packaging also protects the substance 3. The packaging 5 may have a weakened portion to facilitate tearing thereof for its opening.

FIG. 3 illustrates another embodiment of a patch 1 according to the invention. According to this embodiment, the patch 1 comprises spots 6 of hydrophilic powder distributed on the application side of the backing 4. The spots are distributed over a surface coated with substance 3. Therefore, the substance 3 always lies in the vicinity of a spot 6 which guarantees its correct positioning against the skin of a user.

FIG. 4 illustrates another embodiment of a patch 1 according to the invention. In this example, the application side of the backing 4 is coated with a powder 7 comprising a mixture of the hydrophilic powder and the substance. Therefore, uniform adhering of the powder 7 to the skin is ensured, which guarantees homogeneous application of the substance to the skin. In addition, the adhesion surface is thereby optimized, which ensures good holding firm of the patch 1 whilst avoiding local concentration of forces when pulling off the patch 1.

In these different embodiments, it is possible to provide the application side of the backing 4 with electrostatic properties. In this case, the hydrophilic powder 2 (and optionally the substance 3 in powder form) may be held in contact with the application side by electrostatic forces as described in patent EP 1 367 944. Other embodiments of application of the hydrophilic powder 2 can also be considered.

Although the backing 4 illustrated in FIGS. 1 to 4 is flat, other geometries can also be contemplated, notably backings having a recess which forms a chamber, reservoir patches, rigid or semi-rigid backings, flat, or not, and in different types of materials.

In the embodiments illustrated in FIGS. 1 to 4, the patches are approximately comma-shaped. As illustrated in FIG. 5, such a shape of patch 1 is particularly suitable for application to a user's face 8, between the cheek and the nose.

According to one non-illustrated variant, the patch may comprise a peel-off adhesive film above the application side of the backing and therefore lying above the powder (2) and/or substance 3. This peel-off film allows exfoliation of the skin in order to remove part of the stratum corneum. Once the film has been peeled off, the penetration of the applied substance into the body is improved.

The patch 1 may in particular be used for the vaccination of subjects, desensitization of subjects, or to deliver an active substance such as biologically active and/or antigenic (poly)peptides in particular. The patch can therefore be used to vaccinate subjects against any pathogen. Therefore, one particular object of the invention resides in a vaccination method of a subject against a pathogen comprising (i) applying of a patch according to the invention to the skin of a subject, the patch containing an antigen specific to said pathogen, (ii) holding the patch firm for a period of time permitting transfer of the antigen into the skin. Optionally, an exfoliation step may precede the step of holding the patch firm. The pathogen may be of varied type (virus, bacterium, parasite, etc.) and the antigen is typically of polypeptide or lipid type. The patch may also be used to desensitize subjects against allergens. The patch may also be used to deliver any active substance. The substance is typically of polypeptide type, such as a hormone, a cytokine, a growth factor, a trophic factor, etc.

Although the illustrated embodiments detail a patch for the application of an active substance, the invention also applies to a patch devoid of any such substance. Therefore a patch according to the invention may also be solely coated with the hydrophilic powder in order to hydrate the user's skin, in particular for cosmetic applications. The format of said patch is then advantageously occlusive. An occlusive patch is obtained for example by arranging the hydrophilic powder on the periphery of the application side of the backing. As in the embodiment shown in FIG. 3, the hydrophilic powder can also form spots distributed over the application side of the backing. As in the embodiment shown in FIG. 4, the hydrophilic powder can also be arranged over the entire surface of the backing. Said patch may also comprise a sealed pack in which the backing and the hydrophilic powder are contained.

The method of manufacture of a patch according to the invention typically comprises a step of applying a hydrophilic powder onto all or part of the application side of a backing. In one particular embodiment, the method also comprises the application of a biologically active substance onto the application side of the patch backing. Any powdering method known per se can be used. The hydrophilic powder can be arranged on the periphery of the backing, for example so as to surround a surface coated with an active substance. Also, the hydrophilic powder can be applied to the backing to form spots distributed over the application side of the backing. These spots can be formed on a surface coated with the active substance. The areas with the active substance and with the hydrophilic powder can be defined by powdering devices forming different patterns or using different models.

It is also possible to apply a mixture of hydrophilic powder and powder including an active substance onto the application side of the backing. The application of a powder mixture is particularly suitable for patch having a flat backing.

The hydrophilic powder and the active substance can also be applied to the application side of the backing during separate successive steps.

1. A patch for application to the skin, wherein said patch comprises:
a hydrophilic powder that becomes adhesive in the presence of water; and
a backing having an application side on which the hydrophilic powder is deposited.

2. The patch of claim 1, wherein the hydrophilic powder is deposited on a periphery of the application side of the backing.

3. The patch of claim 1, wherein the hydrophilic powder is deposited as spots distributed over the application side of the backing.

4. The patch of claim 1, wherein the hydrophilic powder is deposited on an entire surface of the application side of the backing.

5. The patch of claim 1, wherein said patch further comprises a biologically active substance.

6. The patch of claim 5, wherein the substance comprises a powder mixed with the hydrophilic powder.

7. The patch of claim 5, wherein the substance is deposited on one or more area(s) of the application side of the backing, separate from the hydrophilic powder.

8. The patch of claim 1, wherein the backing is comma-shaped.

9. The patch of claim 1, wherein the hydrophilic powder contains water-soluble polymer chains.

10. The patch of claim 9, wherein the hydrophilic powder comprises at least one of cellulose gum, ethylcellulose, hydroethylcellulose, a copolymer of N-vinyl-2-Pyrrolidone and vinyl acetate, and/or and a polymer of 1-vinyl-2-pyrrolidone.

11. The patch of claim 1, further comprising a reservoir containing an aqueous solution positioned above the hydrophilic powder.

12. The patch of claim 1, further comprising sealed packaging that is impervious to water vapour and in which the backing and the hydrophilic powder are included.

13. A method of manufacture of a patch, the method comprising:
applying a hydrophilic powder onto all or part of an application side of a patch backing, the hydrophilic powder becoming adhesive when hydrated to adhere the patch backing to skin.

14. The method of claim 13, wherein the hydrophilic powder is applied onto the periphery of the application side or is applied in the form of spots distributed over the application side.

15. The method of claim 13, further comprising a step of applying a biologically active substance onto the application side.

16. The method of claim 15, wherein the substance is applied in the form of a powder mixed with the hydrophilic powder.

17. The method of claim 15, wherein the substance and the hydrophilic powder are applied successively.

18. The method of claim 13, wherein the powder is held on the backing via forces of electrostatic type.

19. Use of the patch of claim 1 for the preparation of a product for vaccination, for desensitization or for delivery of any active substance to a subject, or to hydrate skin.
